(19) United States
(12) Patent Application Publication
(10) Pub. No.: US 2025/0366810 A1
(43) Pub. Date: Dec. 4, 2025

(54) DYNAMIC IMAGE ANALYSIS APPARATUS, DYNAMIC IMAGE ANALYSIS METHOD, AND RECORDING MEDIUM

(71) Applicant: Konica Minolta, Inc., Tokyo (JP)

(72) Inventor: Kojiro Ono, Chiba (JP)

(73) Assignee: Konica Minolta, Inc., Tokyo (JP)

(21) Appl. No.: 19/220,532

(22) Filed: May 28, 2025

(30) Foreign Application Priority Data
May 30, 2024 (JP) 2024-087973

(51) Int. Cl. A61B 6/00; A61B 6/50; G06T 7/00; G06T 7/246; G16H 40/63

(52) U.S. Cl. CPC A61B 6/5217; A61B 6/50; G06T 7/0016; G06T 7/246; G16H 40/63; G06T 2207/10024; G06T 2207/20021; G06T 2207/30061

(57) ABSTRACT

A dynamic image analysis apparatus includes an acquisition unit that acquires a dynamic image of an imaging target by dynamic imaging; and a calculation unit that calculates, from the dynamic image, two pieces of displacement amount data related to a region obtained by dividing a still image constituting the dynamic image and change amount data related to a density of the region.

### CROSS REFERENCE TO RELATED APPLICATIONS

Japanese Patent Application No. 2024-087973 filed on May 30, 2024, including description, claims, drawings, and abstract the entire disclosure is incorporated herein by reference in its entirety.

### BACKGROUND

### Technological Field

The present disclosure relates to a dynamic image analysis apparatus, a dynamic image analysis method, and a recording medium.

### Description of Related Art

In dynamic imaging, a radiation generation apparatus repeatedly emits a radiation pulse in a cycle (pulse cycle) a plurality of times per unit time (e.g., 15 times per second) for a predetermined time (duration) while an emission instruction is issued. Then, a radiation detection apparatus reads, as a signal value (intensity), the amount of electric charges generated in accordance with the dose of radiation received through an object. The dynamic imaging captures a dynamic image including a plurality of (a series of) still images taken at imaging times different from one another by the pulse cycle. The cycle at which still images are captured is called the frame rate, and is equal to the cycle of radiation pulses. A doctor diagnoses a disease on the basis of the captured dynamic images. The doctor can make a diagnosis based on the movement of the lungs and the heart by dynamic imaging of organs such as the lungs and the heart. In addition, the doctor can make a diagnosis based on the movement of a joint by dynamic imaging of bones.

As a method of quantitatively evaluating the movement of the lungs such as the lung fields as a whole (macroscopically), a method has been performed in which an indicator value indicating changes in the lung fields is calculated from a dynamic image of the chest and the softness of the lung fields is evaluated on the basis of the calculated indicator value (e.g., Japanese Unexamined Patent Publication No. 2017-176202).

Furthermore, increasing the accuracy in estimating the volume of a moving object in a radiographic image increases the accuracy in estimating an evaluation index for the function of the object that is estimated on the basis of the volume of the object. The volumes of the lung fields are estimated on the basis of extracted frame images for each set, and the respiratory function indices of the lung fields are estimated on the basis of the estimated volumes (e.g., Japanese Unexamined Patent Publication No. 2019-122449).

### SUMMARY

At present, in the medical examination of emphysema (emphysema-type COPD), a respiratory function test is performed in order to diagnose the emphysema and evaluate the degree of progress thereof. In addition, image inspection of chest CT is generally performed as the detailed inspection. Chest CT examination allows confirmation of the degree of progress of emphysema by quantitatively evaluating the area and volume of black areas caused by destruction of lung structures such as alveoli.

However, the CT examination results in higher radiation exposure. In particular, in order to evaluate emphysema in detail, it is necessary to perform CT inspection in two phases of inspiration and expiration, and therefore, the radiation exposure is particularly increased.

It is required to quantitatively evaluate a local part of the lung (microscopically) with respect to deformation of the lung structure accompanying respiratory motion, for example, to evaluate localized deformation and quantification of deformation, by a dynamic image acquired by dynamic imaging with a low exposure dose.

An object of the present invention is to provide a dynamic imaging apparatus, a dynamic image analysis method, and a recording medium capable of providing information for determining the degree of progress of a disease by (microscopically) quantitatively evaluating a local part of a lung using a dynamic image.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a configuration of a dynamic image analysis apparatus;

FIG. 2 is a functional block diagram of a processing circuit;

FIG. 3 is a diagram of a modeled lung structure by making the lung structure correspond to a cube;

FIG. 4A is a diagram illustrating a still image taken from a dynamic image of the lung, in which the lung is in a large state;

FIG. 4B is a diagram illustrating a still image taken from a dynamic image of the lung, in which the lung is in a small state;

FIG. 5A is a diagram illustrating a region resulting from division of a reference image every 50 pixels;

FIG. 5B is a diagram illustrating a state in which a region is displaced in a still image different from a reference image;

FIG. 5C is a diagram illustrating a state in which a region is displaced in a still image different from a reference image;

FIG. 6 is a diagram showing a flowchart for a processing circuit;

FIG. 7 is a diagram illustrating a detailed flowchart for the processing circuit;

FIG. 8 is a diagram showing an example of WIRE and elastic modulus maps displaying the elastic moduli in the X-, Y-, and Z-axes and combined elastic modulus, resulting from smoothing processing for each region;

FIG. 9 is a diagram showing an example of WIRE and strain maps displaying strains in the X-, Y-, and Z-axes and the combined strain, resulting from smoothing processing for each region;

FIG. 10A is a diagram showing a state in which the softness of the lung structure is decreased, the elastic modulus is increased, and the strain ε with respect to the stress σ is decreased;

FIG. 10B is a diagram showing a case where the elastic moduli are different in the X-axis, the Y-axis, and the Z-axis;

FIG. 10C is a diagram in which severities are indicated by slopes;

FIG. 11 is a diagram illustrating a window displaying buttons for a doctor to select a disease to be displayed; and

FIG. 12 is a diagram showing an example in which the elastic moduli E on the third, fourth, and fifth days of hospitalization are displayed.

### DETAILED DESCRIPTION OF EMBODIMENTS

Hereinafter, one or more embodiments of the present invention will be described with reference to the drawings. However, the scope of the invention is not limited to the disclosed embodiments.

### <Dynamic Image Analysis Apparatus>

A configuration of a dynamic image analysis apparatus 100 according to an embodiment of the present disclosure will be described.

### [Configuration]

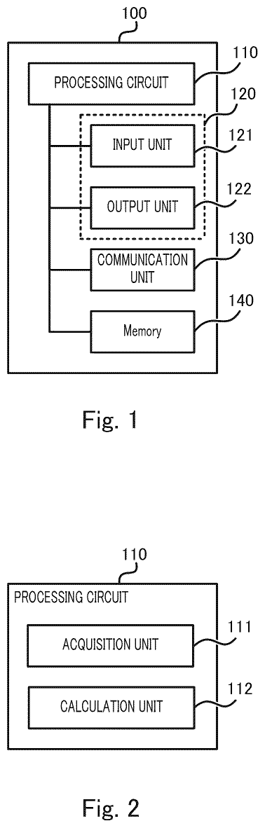

FIG. 1 is a diagram illustrating a configuration of the dynamic image analysis apparatus 100.

The dynamic image analysis apparatus 100 includes a processing circuit 110, an input/output unit 120, a communication unit 130, and a memory 140. The input/output unit 120 includes an input unit 121 and an output unit 122. The input unit 121 and the output unit 122 may be integrally formed. In a case where input and output are performed via the communication unit 130, the input/output unit 120 may be omitted.

The processing circuit 110 is constituted by a central processing unit (CPU), a graphics processing unit (GPU), an application specific integrated circuit (ASIC), a field programmable gate array (FPGA), or the like, and may include a neural network. Based on an input medical image, the processing circuit 110 extracts a feature amount and estimates a disease level of a specific disease. Details of the processing circuit 110 will be described later.

The input unit 121 includes at least one of a touch panel, a keyboard, a mouse, a microphone, and the like, and receives an input based on an operation performed by a user (a doctor, a radiology technician, or the like).

The output unit 122 includes at least one of a display, a speaker, a printer, and the like, and outputs a result determined by the processing circuit 110 to the outside.

The communication unit 130 communicates with an external device via a bus, a local area network (LAN), the Internet, a virtual private network (VPN), a public line, or the like by radio or by wire. The communication unit 130 communicates with a Hospital Information System (HIS), a Radiology Information System (RIS), a Picture Archiving and Communication System (PACS), a dynamic analysis apparatus, and the like.

The memory 140 is constituted by a read only memory (ROM), a random access memory (RAM), an erasable programmable ROM (EPROM), an electrically EPROM (EEPROM), a hard disk drive (HDD), or the like, and stores dynamic images, various programs, and the like.

FIG. 2 is a functional block diagram of the processing circuit 110.

The processing circuit 110 includes an acquisition unit 111 and a calculation unit 112.

The acquisition unit 111 acquires a dynamic image. The dynamic image may be acquired from an external system such as the RIS via the communication unit 130 based on an input from the outside via the input unit 121 or the communication unit 130, or may be acquired from the memory 140. The dynamic image is, for example, a dynamic image obtained by imaging a lung.

The calculation unit 112 calculates the strain and elastic modulus of the imaged lung on the basis of the dynamic image acquired by the acquisition unit 111. Furthermore, the calculated strain and elastic modulus are displayed on the output unit 122 or on an external device via the communication unit 130. Details will be described later.

### [Modeling of Lung]

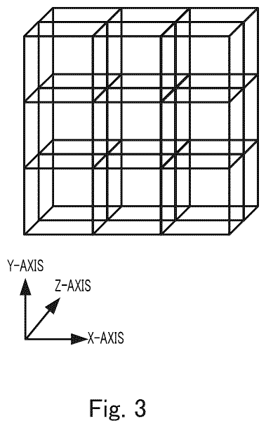

FIG. 3 is a diagram of a modeled lung structure by making the lung structure correspond to a cube. In an actual lung, a larger number of lung structures exist on the X-axis, the Y-axis, and the Z-axis. The dynamic image is captured by a dynamic imaging apparatus repeatedly emitting pulsed radiation in the Z-axis to acquire the transmitted radiation as a still image in the XY plane (Z plane).

First, a change in the size of a lung structure during breathing is considered. Since the lung structure changes in size during breathing, it can be considered that the size of the cube changes in FIG. 3.

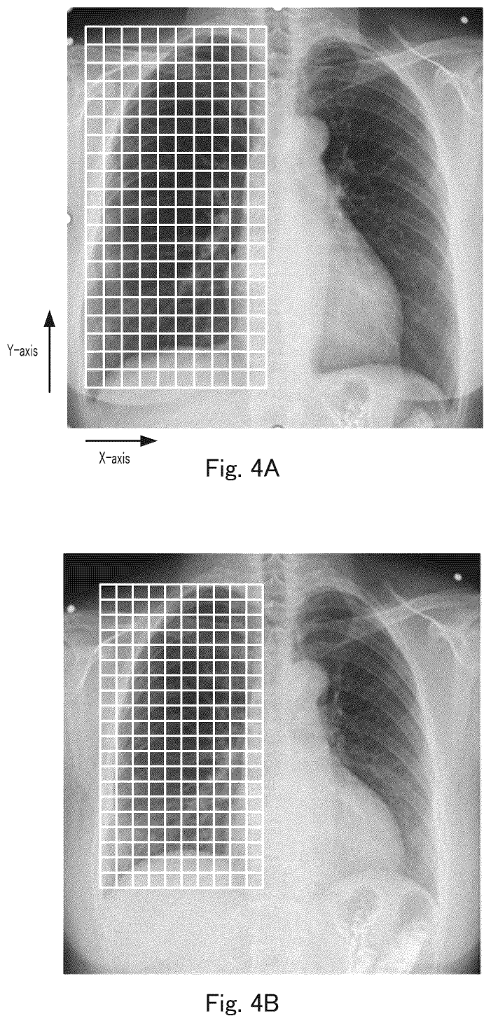

FIGS. 4A and 4B illustrate one still image of the dynamic image of the lungs. For example, FIG. 3 illustrates a state in which radiation is emitted in the Z-axis direction and the XY plane is imaged.

FIG. 4A is a diagram illustrating a still image in a state where the lungs are in a large state (e.g., at maximum inspiration), taken from the dynamic image of the lungs. For example, a state in which the right lung is divided into sections at predetermined intervals. For example, a still image of the expanded lungs is set as a reference image. FIG. 4B is a diagram illustrating a still image in a state in which the lungs are in a small state (e.g., at maximum expiration), taken from the dynamic image of the lungs. A still image of the contracted lungs may be set as the reference image. In FIG. 4B, the same region as the reference image is divided into a plurality of sections, but the size of the entire sections is small because the lungs are in the small state. That is, the lungs change from the large state (FIG. 4A) to the small state (FIG. 4B) with the lapse of time.

Depending on the softness (elastic modulus) of the lungs, the difference in size of the lung structures is different. For example, when the lung structure is in a hard state (having a high elastic modulus), the lung structure does not contract much, so that the difference between the size of the lung structure in a large state and the size of the lung structure in a small state is small. When the lung structure is in a soft state (where the elastic modulus is low), the lung structure contracts significantly, so that the difference in size between the size in the large state of the lung structure and the size in the small state of the lung structure is large.

Therefore, the softness of the lung structure can be grasped by comparing the sizes of the lungs in a series of still images constituting the dynamic image.

Thus, when FIGS. 4A and 4B are compared, the softness of the lung structure on the X-axis and the Y-axis can be grasped, but the softness of the lung on the Z-axis cannot be grasped.

For example, in interstitial pneumonia, the lung structure becomes fibrotic, resulting in a state in which the lung contracts in one direction but does not contract in another direction. Therefore, it is necessary to grasp the softness of the lung structure in all three dimensional directions.

### [Elastic Modulus]

The stresses that deform the object are defined as nine stress components of σₓₓ, σ_{yy}, σ_{zz}, σ_{xy}, σ_{xz}, σ_{yx}, σ_{yz}, σ_{zx}, and σ_{zy}. σₓₓ, σ_{yy}, and σ_{zz} are normal stresses to the X plane (YZ plane), the Y plane (XZ plane), and the Z plane (XY plane) respectively. σ_{xy}, σ_{xz}, σ_{yx}, σ_{yz}, σ_{zx}, σ_{zy} are shear stresses.

[1]

$$σ = ( \begin{matrix}{σ}_{xx} & {σ}_{xy} & {σ}_{xz} \\ {σ}_{yx} & {σ}_{yy} & {σ}_{yz} \\ {σ}_{zx} & {σ}_{zy} & {σ}_{zz}\end{matrix} ) \qquad ( 1 )$$

According to Hooke's law, the elastic modulus E has a relationship of σ=Eε with respect to the stress σ and the strain ε. That is, in terms of the stress components,

[2]

$$( \begin{matrix}{σ}_{xx} & {σ}_{xy} & {δ}_{xz} \\ {σ}_{yx} & {σ}_{yy} & {σ}_{yz} \\ {σ}_{zx} & {σ}_{zy} & {σ}_{zz}\end{matrix} ) = E ( \begin{matrix}{ε}_{xx} & {ε}_{xy} & {ε}_{xz} \\ {ε}_{yx} & {ε}_{yy} & {ε}_{yz} \\ {ε}_{zx} & {ε}_{zy} & {ε}_{zz}\end{matrix} ) . \qquad ( 2 )$$

Since only the normal strains need to be considered for the change in the size of the lung structure, only the normal stresses are considered and the shear stresses may be set to zero. Therefore, since, in the present disclosure,

[3]

$$σ = ( \begin{matrix}{σ}_{xx} & 0 & 0 \\ 0 & {σ}_{yy} & 0 \\ 0 & 0 & {σ}_{zz}\end{matrix} ) \qquad ( 3 )$$

may hold true,
[4]

$$( \begin{matrix}{σ}_{xx} & 0 & 0 \\ 0 & {σ}_{yy} & 0 \\ 0 & 0 & {σ}_{zz}\end{matrix} ) = E ( \begin{matrix}{ε}_{xx} & 0 & 0 \\ 0 & {ε}_{yy} & 0 \\ 0 & 0 & {ε}_{zz}\end{matrix} ) \qquad ( 4 )$$

only needs to be considered.

Since the strains εₓₓ, ε_{yy}, and ε_{zz} are strains in the X-axis, the Y-axis, and the Z-axis, respectively, they are the amounts of change Δₓ, Δ_{y}, and Δ_{z} in the size of the lung structure.

Therefore, if the stresses σₓₓ, σ_{yy}, and σ_{zz} and the displacement amounts Δₓ, Δ_{y}, and Δ_{z} in the X-axis, the Y-axis, and the Z-axis are known, the elastic moduli Eₓ, E_{y}, and E_{z} can be obtained.

### [Displacement Amount]

In dynamic imaging of the lungs, a change in images is presumed to be due to a change in the lung structure. Changes in the lung structure can be measured by detecting changes in the images based on an optical flow.

Since the dynamic imaging includes a plurality of still images, the displacement amounts (Δₓ and Δ_{y}) of the lungs in the X-axis and Y-axis directions can be calculated by comparing the positions of the lungs in the still images.

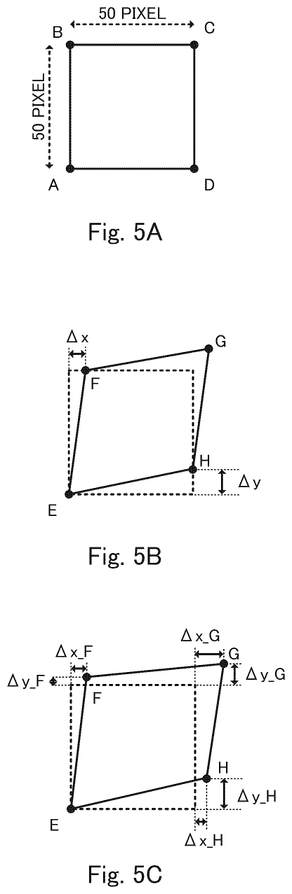

First, one of the still images forming the dynamic image is set as a reference image. FIG. 5A is a diagram illustrating a region ABCD obtained by dividing the reference image, for example, every 50 pixels. FIG. 5B is a diagram illustrating a state in which the region ABCD is displaced to a region EFGH in a still image different from the reference image. The size for the division may be a size corresponding to the size of one lung structure, may be another size, or may be set according to processing capacity and image quality.

In the present disclosure, strain is obtained as a change in the size and shape of the region. Since the strain is a change in the positional relationship between the points, it is only necessary to determine how much the points F, G, and H with respect to the point E are displaced from the positions of the points B, C, and D with respect to the point A. Since the region ABCD is a minute region, the region EFGH may be approximated as a parallelogram, and Δₓ and Δ_{y} may be obtained.

FIG. 5C illustrates an example in which a displacement is obtained for each of the point F, the point G, and the point H. An average of displacements of the point F, the point G, and the point H with respect to the X-axis and an average of displacements thereof with respect to the Y-axis may be obtained as Δₓ and Δ_{y}. In the case of displacements in FIG. 5B, the same values as Δₓ and Δ_{y} obtained in FIG. 5B are obtained by dividing the total of the displacements of the points E, F, G, and H by 2.

The reference image may be the very first still image among a series of still images constituting the dynamic image.

As described above, when the region ABCD is displaced to the region EFGH, the displacement amounts Δₓ and Δ_{y} can be obtained.

Meanwhile, Δ_{z} can be obtained from a change in density.

There are capillaries on the surface of the lung structure, and blood flows through the capillaries. With the change in the size of the lung structure, the density of capillaries changes. The smaller the lung structure, the higher the capillary density, and the larger the lung structure, the lower the capillary density. The change in the density of the capillaries means a change in the density of blood, and the change in the density of blood is detected as a change in the density of the still image (rate of change or density difference). The density of the region ABCD may be calculated as an average of the densities of the entire region or may be calculated as an average of the densities at the point A, the point B, the point C, and the point D.

By detecting the change in the density of the region ABCD, the displacement amount Δ_{z} in the Z-axis direction can be calculated. The density of a still image is the reception intensity of radiation, and therefore the amount of electric charge (signal value) detected by a radiation detection apparatus including a plurality of detection elements may be used as the density. Assuming that the lung structure is spherical and the size in the Z-axis direction is the same as those in the X-axis and the Y-axis in the reference image, the density of the reference image is a density in the case of 50 pixels in the Z-axis direction, and thus the displacement amount Δ_{z} can be obtained from the rate of change.

Since the displacement amount Δ is the strain ε, the elastic modulus E can be obtained according to Hooke's law (σ=Eε), if the stress σ can be obtained.

### [Stress]

The respiratory motion is motion of a fluid called air. Since the change in the size of the lung structure is based on the respiratory motion, the change in the size of the lung structure is based on the dynamic pressure. According to the Bernoulli's theorem, a dynamic pressure q satisfies q=pv²/2 with respect to the density ρ and the fluid velocity v.

In the present disclosure, since the dynamic pressure q is the stress σ, the density ρ is the gas density P, and the fluid velocity v is the movement velocity V, σ=pv²/2 holds true.

Since the stresses σₓₓ, σ_{yy}, and σ_{zz} are normal stresses on the X plane, the Y plane, and the Z plane, they are stresses along the X-axis, the Y-axis, and the Z-axis, respectively. Similarly, Pₓ, P_{y}, P_{z} obtained by decomposing the gas densities P in the X-axis, the Y-axis, and the Z-axis and Vₓ, V_{y}, V_{z} obtained by decomposing the movement velocities V in the X-axis, the Y-axis, and the Z-axis satisfy the following relationships:

$${σ}_{xx} = {P}_{x} {V}_{x}^{2} / 2 ; \\ {σ}_{yy} = {P}_{y} {V}_{y}^{2} / 2 ; \\ {σ}_{zz} = {P}_{z} {V}_{z}^{2} / 2.$$

In the present disclosure, the lung structure becomes large when air enters the lungs, and the lung structure becomes small when air comes out of the lungs. That is, a change in the lung structure occurs due to the stress of the air. Since the stresses due to the air are equal in the X-axis, the Y-axis, and the Z-axis, σₓₓ=σ_{yy}=σ_{zz} is obtained.

Since the gas density P of the air is 1, the stresses σₓₓ, σ_{yy}, and σ_{zz} can be obtained if the movement velocity V of the air is known.

For example, when a patient wears a ventilator, the movement velocity V of air is a flow velocity set in the ventilator. Furthermore, it is also possible to detect the amount of movement of the diaphragm on the basis of the dynamic images and obtain the movement velocity V of the air on the basis of the amount of movement of the diaphragm. In this way, since the movement velocity V of the air of each of the expiration and the inspiration can be obtained, the stress σ can be obtained.

### [Processing]

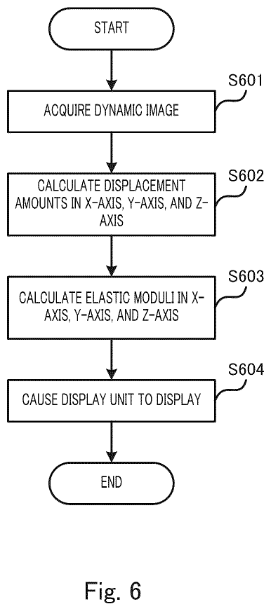

FIG. 6 is a diagram illustrating a flowchart of the processing circuit 110.

The acquisition unit 111 acquires a dynamic image (step S601).

The calculation unit 112 calculates the displacement amounts in the X-axis and the Y-axis based on the dynamic image acquired by the acquisition unit 111. In addition, the calculation unit 112 calculates the displacement amount in the Z-axis based on the density difference between the still images constituting the dynamic image (step S602).

The calculation unit 112 calculates the elastic moduli in the X-axis, the Y-axis, and the Z-axis from the displacement amounts in the X-axis, the Y-axis, and the Z-axis (step S603).

The calculation unit 112 causes the output unit 122 to display the displacement amounts and the elastic moduli in the X-, Y-, and Z-axes calculated by the calculation unit 112 (step S604). Instead of the calculation unit 112, a display control unit (not illustrated) in the processing circuit 110 may cause a display unit to perform the display. The displacement amounts in the X-axis, the Y-axis, and the Z-axis may be displayed between step S602 and S603.

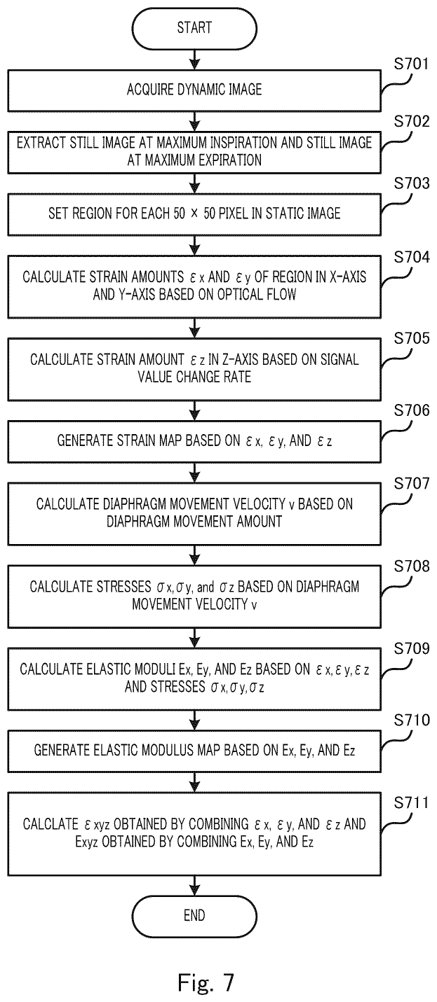

FIG. 7 is a diagram illustrating a detailed flowchart of the processing circuit 110.

The acquisition unit 111 acquires a dynamic image (step S701).

The calculation unit 112 extracts a still image of maximum inspiration and a still image of maximum expiration from the dynamic image acquired by the acquisition unit 111 (step S702).

The calculation unit 112 determines one of the still image of maximum inspiration and the still image of maximum expiration as a reference image, and sets a region for each 50×50 pixel in the reference image (step S703).

The calculation unit 112 calculates the strains εₓ and ε_{y} in the X-axis and the Y-axis for each region based on the optical flow with respect to the reference image (step S704).

The calculation unit 112 calculates the strain ε_{z} in the Z-axis based on the change rate of the signal value (the amount of electric charge) for each region (step S705). Assuming that the lung structure is spherical and the size in the Z-axis direction is the same as those in the X-axis and the Y-axis in the reference image, the density of the reference image is a density in the case of 50 pixels in the Z-axis direction, and thus the strain E_{z} can be calculated from the rate of change.

The calculation unit 112 generates a strain map on the basis of εₓ and ε_{y} calculated in step S704 and ε_{z} calculated in step S705 (step S706). The strain map is a map showing the strain εₓ in the X-axis, the strain ε_{y} in the Y-axis, and the strain ε_{z} in the Z-axis for each region.

The calculation unit 112 calculates the amount of movement of the diaphragm based on the dynamic image. The calculation unit 112 calculates the diaphragmatic movement velocity v on the basis of the calculated amount of movement and the frame rate of the dynamic image (step S707). The calculation unit 112 may obtain a flow rate set for the ventilator instead of calculating the movement velocity v of the diaphragm. The flow rate set for the ventilator may be acquired from the ventilator via the communication unit 130, may be acquired from an electronic medical record of the patient, may be acquired from the memory 140, or may be acquired by other means.

The calculation unit 112 calculates the stresses σₓ, σ_{y}, and σ_{z} from the diaphragmatic movement velocity v or the flow rate set for the ventilator (step S708).

The calculation unit 112 calculates the elastic moduli Eₓ, E_{y}, and E_{z} based on the strains εₓ, ε_{y}, and ε_{z} calculated in step S704 and step S705 and the stresses σₓ, σ_{y}, and σ_{z} calculated in step S708 (step S709).

The calculation unit 112 generates an elastic modulus map on the basis of the elastic moduli Eₓ, E_{y}, and E_{z} calculated in step S709 (step S710). The elastic modulus map is a map in which the elastic modulus Eₓ in the X-axis, the elastic modulus E_{y} in the Y-axis, and the elastic modulus E_{z} in the Z-axis are illustrated for each region.

The calculation unit 112 calculates the combined strain ε_{xyz} based on the strains εₓ, ε_{y}, and ε_{z}. Further, the calculation unit 112 calculates the combined elastic modulus E_{xyz} based on the elastic moduli Eₓ, E_{y}, and E_{z} (step S711). The calculated combined strain ε_{xyz} and combined modulus E_{xyz} are calculated as a map.

### [Display]

The elastic modulus E can be obtained by obtaining the strain ε (displacement amount Δ) and the stress σ.

Since the strain ε and the stress σ are obtained for each of the regions ABCD obtained by dividing the reference image in the dynamic image, the elastic modulus E is obtained for each of the divided regions.

The obtained strains ε in the X-axis, the Y-axis, and the Z-axis are displayed for each region (strain map).

In addition, the obtained elastic moduli Eₓ, E_{y}, and E_{z} in the X-axis, the Y-axis, and the Z-axis are displayed for each region (elastic modulus map).

The calculated strains or elastic moduli may be normalized by setting the strain or elastic modulus of a normal lung structure to 1. Normalization using the strain or elastic modulus of the normal lung structure set to 1 makes it possible to grasp the severity of the lung structure easily.

The color for the strains or the elastic moduli to be displayed may be changed for each of the X-axis, the Y-axis, and the Z-axis. For example, displaying in red may be used for the X-axis, in green for the Y-axis, and in blue for the Z-axis. The color (density) may be changed according to the value of the strain or the elastic modulus of each region. For example, a region where the strain or elastic modulus in the X-axis is high may be displayed in dark red (for example, a scarlet color), and a region where the strain or elastic modulus in the X-axis is low may be displayed in light red (for example, a pink color). For example, a region where the strain or the elastic modulus in the Y-axis is high may be displayed in dark green (e.g., forest green), and a region where the strain or the elastic modulus in the Y-axis is low may be displayed in light green (e.g., spring green). For example, a region where the strain or elastic modulus in the Z-axis is high may be displayed in dark blue (for example, indigo blue), and a region where the strain or elastic modulus in the Z-axis is low may be displayed in light blue (for example, sky blue).

A strain or elastic modulus (combined elastic modulus) obtained by combining the strains or elastic moduli in the X-axis, the Y-axis, and the Z-axis may be displayed. For example, the combined strain or elastic modulus may be displayed in purple, a region where the combined strain or elastic modulus is high may be displayed in dark purple (for example, dark color), and a region where the combined strain or elastic modulus is low may be displayed in light purple (for example, mauve color). The combination may be a vector combination, or an average of the strains or elastic moduli in the X-axis, the Y-axis, and the Z-axis (the sum is divided by 3) may be calculated.

An elastic modulus obtained by averaging among regions belonging to the upper lobe, an elastic modulus obtained by averaging among regions belonging to the middle lobe, and an elastic modulus obtained by averaging among regions belonging to the lower lobe may be calculated, and the elastic moduli may be displayed in an overlapping manner at corresponding positions.

Together with the display for each region, a result of smoothing processing performed on the region may be displayed.

By displaying the strain or the elastic modulus for each region, it is possible to grasp the medical condition for each part of the lung.

In addition, WIRE indicating how regions obtained by dividing the reference image every 50 pixels in the XY plane are strained may be displayed. A target still image on which WIRE is displayed may be a still image in which the maximum strain occurs.

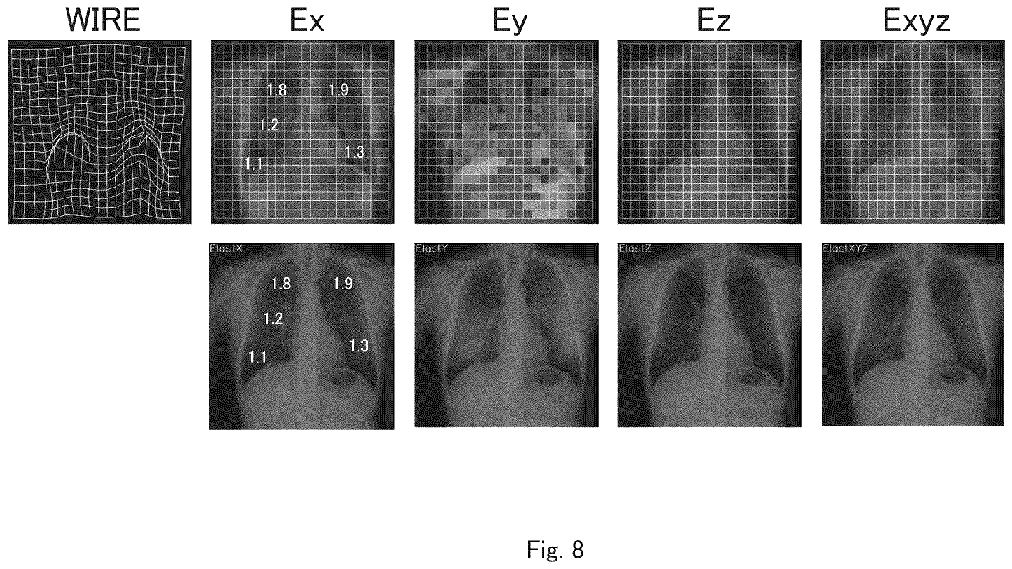

FIG. 8 is a diagram showing an example of WIRE and elastic modulus maps displaying the elastic moduli in the X-, Y-, and Z-axes and composite elastic modulus, resulting from smoothing processing for each region. The average elastic modulus may not be indicated if necessary. WIRE represents strain as it is, and a strained state as illustrated in FIG. 5C is represented as it is. The upper part shows a display for each region, and the lower part shows the results of smoothing processing. FIG. 8 shows an example in which the elastic modulus obtained by averaging among the regions belonging to the upper lobe, the elastic modulus obtained by averaging among the regions belonging to the middle lobe, and the elastic modulus obtained by averaging among the regions belonging to the lower lobe are displayed in an overlapping manner with respect to the X-axis. The average elastic modulus of the upper lobe, the middle lobe, and the lower lobe may be displayed in an overlapping manner also with respect to the elastic moduli in the Y-axis and the Z-axis and the combined elastic modulus. The average elastic moduli for overlap display may be elastic moduli normalized with the elastic modulus of a normal lung structure being 1.

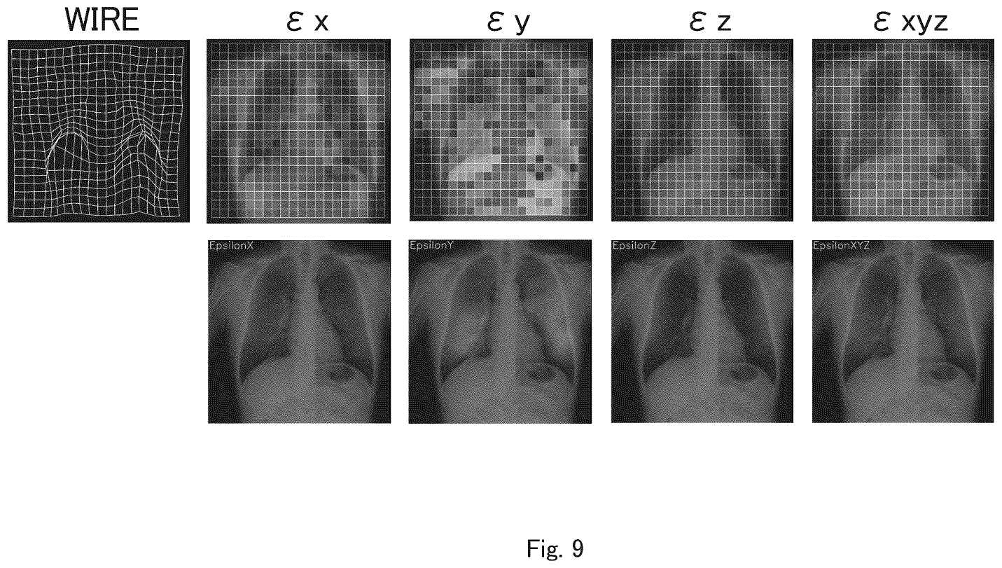

FIG. 9 is a diagram showing an example of WIRE and strain maps displaying strains in the X-, Y-, and Z-axes, resulting from smoothing processing for each region. The upper part shows a display for each region, and the lower part shows the results of smoothing processing. The average strains of the upper lobe, the middle lobe, and the lower lobe in the X-axis, the Y-axis, and the Z-axis may be displayed in an overlapping manner.

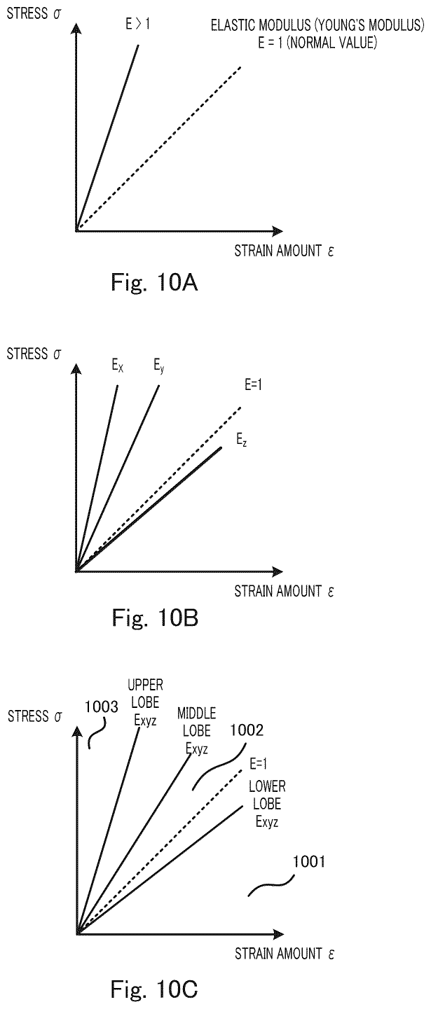

FIGS. 10A to 10C are diagrams showing the relationship between strain ε and stress σ. In FIGS. 10A to 10C, the elastic modulus in the case of a normal lung (lung structure) is normalized to 1. The normalization may be an average of elastic moduli in a case where the lung structure is normal, or may be an upper limit elastic modulus. FIG. 10A is a view showing a state where the softness of the lung structure is decreased, the elastic modulus is increased, and the strain ε with respect to the stress σ is decreased. FIG. 10B is a diagram showing a case where the elastic moduli are different in the X-axis, the Y-axis, and the Z-axis. The severity of the disease can be determined from the slope of the graph (the magnitude of the elastic modulus). FIG. 10C shows a diagram in which severities are indicated by slopes. For example, a region 1001 indicates a normal range, a region 1002 indicates a mild range, and a region 1003 indicates a severe range. For example, the region 1001 may be displayed in blue, the region 1002 may be displayed in yellow, and the region 1003 may be displayed in red. The number of regions is not limited to three. The ranges of severe/mild/normal may be set according to the disease. In FIGS. 10A to 10C, the elastic modulus may be displayed for each of the upper lobe, the middle lobe, and the lower lobe. FIG. 10C shows that the upper lobe is severe, the middle lobe is mild, and the lower lobe is normal. FIGS. 10A to 10C may be displayed in a portion below WIRE in FIGS. 8 and 9.

### [Disease]

The patient has been diagnosed by other means as to what kind of disease it is, for example, COPD, interstitial pneumonia, or pneumothorax. The doctor can cause the output unit 122 to perform display corresponding to the disease by inputting the disease of the patient from the input unit 121.

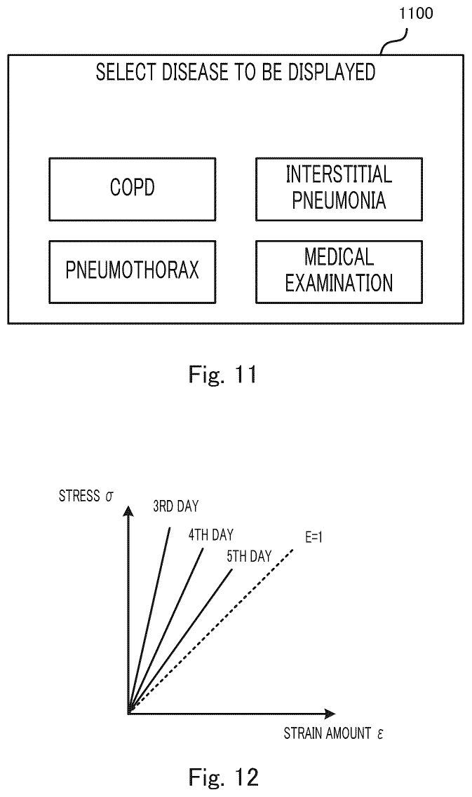

FIG. 11 is a diagram illustrating a window 1100 displaying buttons for the doctor to select a disease to be displayed. In the window 1100, some of the diseases may not be displayed, other diseases may be displayed, and the number of displayed buttons may not be four. For example, when a medical examination is input, the dynamic image analysis apparatus 100 may provide a display for a plurality of diseases.

### [COPD]

In patients with COPD, it is possible to observe clusters of multiple lung structures that have undergone emphysematous changes—that is, Air Trapping, where air cannot be expelled from the lung structures. Since Air-Trapping causes the lung structure not to contract, the softness decreases, that is, the elastic modulus increases. That is, as the elastic modulus increases (softness decreases), it can be determined that the condition of COPD is more serious.

Therefore, a doctor can diagnose the condition of COPD in a patient with COPD by the displays of FIGS. 8, 9, and 10A to 10C.

### [Interstitial Pneumonia]

For patients with interstitial pneumonia, fibrosis of the lung structure can be observed. The fibrosis of the lung structure makes the lung structure less likely to contract in a certain direction. That is, in the dynamic image of an interstitial pneumonia patient, as illustrated in FIG. 10B, there are differences in softness (elastic modulus) among the X-axis, the Y-axis, and the Z-axis.

Therefore, for a patient with interstitial pneumonia, a doctor can grasp the medical condition of interstitial pneumonia from the display of FIGS. 8, 9, and 10B.

### [Pneumothorax]

For a patient with pneumothorax (lung collapse), it is possible to observe a situation in which air leaks out into the pleural cavity to compress the lungs, and the lungs shrink. Therefore, since the strain ε of the lung structure of the compressed portion decreases, the elastic modulus increases.

Therefore, a doctor can diagnose the medical condition of pneumothorax for a pneumothorax patient by the displays of FIGS. 8, 9, and 10.

### [Treatment Situation]

In dynamic imaging, a patient (person to be imaged) is exposed to a small amount of radiation, and therefore dynamic imaging can be performed on the patient a large number of times. Therefore, by comparing the elastic moduli E according to the situation of the treatment, it is possible to know the improvement situation of the disease due to the effect of the treatment. FIG. 12 is a diagram showing an example in which the elastic moduli E on the third, fourth, and fifth days of hospitalization are displayed. The elastic modulus E to be displayed may be any of the Eₓ, E_{y}, E_{z}, and E_{xyz}, or may be a plurality of elastic moduli. In a case where the dynamic image analysis apparatus 100 displays a plurality of elastic moduli, for example, the dynamic image analysis apparatus 100 may display the elastic moduli by changing the color for each of the third, fourth, and fifth days of hospitalization, or may display the elastic moduli by changing the color for each of Eₓ, E_{y}, E_{z}, and E_{xyz}. The doctor can determine, from the display in FIG. 12, whether the condition of the patient is improving. The doctor can determine a future treatment policy by understanding the improvement of the condition of the disease.

Although the embodiments have been described above with reference to the drawings, the present disclosure is not limited to such examples. It is obvious that a person skilled in the art can conceive of various change examples or modification examples within the scope described in the claims. It is to be understood that such changes or modifications also belong to the technical scope of the present disclosure. Furthermore, the constituent elements in the embodiments may be combined as appropriate without departing from the spirit of the present disclosure.
(1) A dynamic image analysis apparatus according to an embodiment of the present disclosure includes: an acquisition unit that acquires a dynamic image of an imaging target by dynamic imaging; and a calculation unit that calculates, from the dynamic image, two pieces of displacement amount data related to a region obtained by dividing a still image constituting the dynamic image and change amount data related to a density of the region.
(2) An image determination apparatus according to an embodiment of the present disclosure is the image determination apparatus of (1) in which the density is a signal value of the dynamic image.
(3) An image determination apparatus according to an embodiment of the present disclosure is the image determination apparatus of (2) in which the two pieces of displacement amount data relating to the region are strain in an X-axis direction and strain in a Y-axis direction in which the region is strained over time, and the change amount data relating to the density of the dynamic image is a change rate of an electric charge detected by a radiation detection apparatus over time.
(4) An image determination apparatus according to an embodiment of the present disclosure is the image determination apparatus of (3) in which the calculation unit calculates strain in a Z-axis direction based on the change rate of the electric charge.
(5) An image determination apparatus according to an embodiment of the present disclosure is the image determination apparatus of (4) in which the calculation unit obtains an elastic modulus in the X-axis direction based on the strain in the X-axis direction, an elastic modulus in the Y-axis direction based on the strain in the Y-axis direction, and an elastic modulus in the Z-axis direction based on the strain in the Z-axis direction.
(6) An image determination apparatus according to an embodiment of the present disclosure is the image determination apparatus of (5) in which the imaging target is an organ.
(7) An image determination apparatus according to an embodiment of the present disclosure is the image determination apparatus of (6) in which the lung is an organ.
(8) An image determination apparatus according to an embodiment of the present disclosure is the image determination apparatus of (4) in which the calculation unit causes a display unit to display the strain in the X-axis direction, the strain in the Y-axis direction, and the strain in the Z-axis direction in different colors.
(9) An image determination apparatus according to an embodiment of the present disclosure is the image determination apparatus of (8) in which the calculation unit causes the strain in the X-axis direction, the strain in the Y-axis direction, and the strain in the Z-axis direction to be displayed at densities corresponding respectively to the strains.
(10) An image determination apparatus according to an embodiment of the present disclosure is the image determination apparatus of (4) in which the calculation unit calculates strain obtained by combining the strain in the X-axis direction, the strain in the Y-axis direction, and the strain in the Z-axis direction.
(11) An image determination apparatus according to an embodiment of the present disclosure is the image determination apparatus of (5) in which the calculation unit causes a display unit to display the elastic modulus in the X-axis direction, the elastic modulus in the Y-axis direction, and the elastic modulus in the Z-axis direction in different colors.
(12) An image determination apparatus according to an embodiment of the present disclosure is the image determination apparatus of (11) in which the calculation unit causes the elastic modulus in the X-axis direction, the elastic modulus in the Y-axis direction, and the elastic modulus in the Z-axis direction to be displayed at densities corresponding respectively to the elastic moduli.
(13) An image determination apparatus according to an embodiment of the present disclosure is the image determination apparatus of (5) in which the calculation unit calculates an elastic modulus obtained by combining the elastic modulus in the X-axis direction, the elastic modulus in the Y-axis direction, and the elastic modulus in the Z-axis direction.
(14) An image determination apparatus according to an embodiment of the present disclosure is the image determination apparatus of (5) in which the calculation unit causes a normal elastic modulus and a calculated elastic modulus to be displayed.
(15) An image determination apparatus according to an embodiment of the present disclosure is the image determination apparatus of (5) in which the calculation unit causes elastic moduli of a plurality of the dynamic images to be displayed.
(16) A dynamic image analysis method according to an embodiment of the present disclosure includes: acquiring a dynamic image of an imaging target by dynamic imaging; and calculating, from the dynamic image, two pieces of displacement amount data related to a region obtained by dividing a still image constituting the dynamic image and change amount data related to a density of the region.
(17) A dynamic image analysis program according to an embodiment of the present disclosure causes a computer to execute: acquiring a dynamic image of an imaging target by dynamic imaging; and calculating, from the dynamic image, two pieces of displacement amount data related to a region obtained by dividing a still image constituting the dynamic image and change amount data related to a density of the region.

Although embodiments of the present invention have been described and illustrated in detail, it is clearly understood that the same is by way of illustration and example only and not limitation, the scope of the present invention should be interpreted by terms of the appended claims.

What is claimed is:

1. A dynamic image analysis apparatus, comprising:
a hardware processor, wherein the hardware processor
acquires a dynamic image of an imaging target by dynamic imaging, and
calculates, from the dynamic image, two pieces of displacement amount data relating to a region obtained by dividing a still image constituting the dynamic image and change amount data relating to a density of the region.

2. The dynamic image analysis apparatus according to claim 1, wherein
the density is a signal value of the dynamic image.

3. The dynamic image analysis apparatus according to claim 2, wherein:
the two pieces of displacement amount data relating to the region are strain in an X-axis direction and strain in a Y-axis direction in which the region is strained over time, and
the change amount data relating to the density of the dynamic image is a change rate of an electric charge detected by a radiation detection apparatus over time.

4. The dynamic image analysis apparatus according to claim 3, wherein
the hardware processor calculates strain in a Z-axis direction based on the change rate of the electric charge.

5. The dynamic image analysis apparatus according to claim 4, wherein
the hardware processor obtains an elastic modulus in the X-axis direction based on the strain in the X-axis direction, an elastic modulus in the Y-axis direction based on the strain in the Y-axis direction, and an elastic modulus in the Z-axis direction based on the strain in the Z-axis direction.

6. The dynamic image analysis apparatus according to claim 5, wherein
the imaging target is an organ.

7. The dynamic image analysis apparatus according to claim 6, wherein
the organ is a lung.

8. The dynamic image analysis apparatus according to claim 4, wherein
the hardware processor causes a display unit to display the strain in the X-axis direction, the strain in the Y-axis direction, and the strain in the Z-axis direction in different colors.

9. The dynamic image analysis apparatus according to claim 8, wherein
the hardware processor causes the strain in the X-axis direction, the strain in the Y-axis direction, and the strain in the Z-axis direction to be displayed at densities corresponding respectively to the strains.

10. The dynamic image analysis apparatus according to claim 4, wherein
the hardware processor calculates strain obtained by combining the strain in the X-axis direction, the strain in the Y-axis direction, and the strain in the Z-axis direction.

11. The dynamic image analysis apparatus according to claim 5, wherein
the hardware processor causes a display unit to display the elastic modulus in the X-axis direction, the elastic modulus in the Y-axis direction, and the elastic modulus in the Z-axis direction in different colors.

12. The dynamic image analysis apparatus according to claim 11, wherein
the hardware processor causes the elastic modulus in the X-axis direction, the elastic modulus in the Y-axis direction, and the elastic modulus in the Z-axis direction to be displayed at densities corresponding respectively to the elastic moduli.

13. The dynamic image analysis apparatus according to claim 5, wherein
the hardware processor calculates an elastic modulus obtained by combining the elastic modulus in the X-axis direction, the elastic modulus in the Y-axis direction, and the elastic modulus in the Z-axis direction.

14. The dynamic image analysis apparatus according to claim 5, wherein
the hardware processor causes a normal elastic modulus and a calculated elastic modulus to be displayed.

15. The dynamic image analysis apparatus according to claim 5, wherein
the hardware processor causes elastic moduli of a plurality of the dynamic images to be displayed.

16. A dynamic image analysis method for an information processing apparatus, comprising:
acquiring a dynamic image of an imaging target by dynamic imaging; and
calculating, from the dynamic image, two pieces of displacement amount data related to a region obtained by dividing a still image constituting the dynamic image and change amount data related to a density of the region.

17. A non-transitory computer-readable recording medium storing a dynamic image analysis program for causing a computer to execute:
acquiring a dynamic image of an imaging target by dynamic imaging; and
calculating, from the dynamic image, two pieces of displacement amount data related to a region obtained by dividing a still image constituting the dynamic image and change amount data related to a density of the region.
